(19) United States
(12) Patent Application Publication
(10) Pub. No.: US 2025/0352385 A1
(43) Pub. Date: Nov. 20, 2025

(54) OSTOMY BARRIER RELEASE LINER SYSTEM

(71) Applicant: Hollister Incorporated, Libertyville, IL (US)

(72) Inventor: Mark W. Jockel, Vernon Hills, IL (US)

(73) Assignee: Hollister Incorporated, Libertyville, IL (US)

(21) Appl. No.: 19/119,559

(22) PCT Filed: Nov. 13, 2023
(86) PCT No.: PCT/US2023/079471

§ 371 Date: Apr. 9, 2025

Related U.S. Application Data
(60) Provisional application No. 63/425,015, filed on Nov. 14, 2022.

(51) Int. Cl. A61F 5/443; A61M 25/02

(52) U.S. Cl. CPC A61F 5/443; A61M 25/02; A61F 5/445; A61F 5/448; A61F 13/0253; A61F 13/0259; A61M 2025/0266; A61M 2205/586

(57) ABSTRACT

An ostomy barrier assembly for attaching an ostomy appliance to a peristomal skin surrounding a stoma includes a skin barrier, an inlet opening, and a two-piece split release liner. The skin barrier may include an adhesive. The inlet opening may be defined in the skin barrier for receiving the stoma. The two-piece split release liner may be connected to the skin barrier, may include at least one finger hole, and may be configured to expose the adhesive to the peristomal skin while the skin barrier is aligned with and surrounds the stoma.

### BACKGROUND

This disclosure is related to an ostomy barrier. More particularly, the present disclosure pertains to an ostomy barrier release liner system.

Ostomy pouches for collecting bodily waste are used by individuals who have had surgery such as a colostomy, ileostomy, or urostomy. An ostomy pouch may be attached to a user via an ostomy barrier, which is configured to seal against peristomal skin surfaces and protect the peristomal surfaces from exposure to stomal effluent. However, the topography of stomas and peristomal surfaces surrounding stomas varies among patients, and sealing an ostomy appliance against such different peristomal surfaces and stomas remains an area for further improvement. For example, an improper fit of the ostomy barrier to the skin may leave gaps or channels that may cause premature leakage.

A person applying an ostomy may find that aligning the ostomy barrier around the stoma and preparing the peristomal skin before securing the barrier to the skin may provide a better fit that prevents premature leakage. However, ostomy barriers require a liner to be removed before aligning the barrier around the stoma and securing the barrier to the skin. It has been found that, at times, even the most careful individuals may misalign the barrier and not properly prepare the peristomal skin surface. This may occur, for example, unless the barrier is partially applied while aligning the barrier to the stoma.

Accordingly, it is desirable to provide an improved ostomy barrier appliance that allows alignment of the barrier around the stoma and preparation of the peristomal skin.

### BRIEF SUMMARY

In one aspect, an adhesive device may include an adhesive for attachment to skin and a two-piece release liner system for protecting the adhesive. The two-piece release liner system may include a first release liner and a second release liner. The first release liner may include a first contact portion configured to be attached to the adhesive and a first grasping portion including at least one finger hole configured to facilitate removal of the first release liner by a user.

In an embodiment, the adhesive device may be an ostomy barrier assembly for attaching an ostomy appliance to a peristomal skin surrounding a stoma. The ostomy barrier assembly may include a skin barrier comprising the adhesive, an inlet opening defined in the skin barrier for receiving the stoma, and the two-piece release liner system attached to the skin barrier. The two-piece release liner system may be configured to allow a user to expose the adhesive to the peristomal skin while the skin barrier is aligned with and surrounds the stoma.

In an embodiment, the two-piece release liner system may be configured such that the first release liner covers about a half of the adhesive and the second release liner covers the other half of the adhesive. In such an embodiment, a split between the first and second release liners may be defined near a center of the adhesive. In some embodiments, the two-piece release liner system may include a center opening aligned with the inlet opening.

In an embodiment, at least one of the first and second release liners may be configured to extend past a periphery of the adhesive. In some embodiments, the second release liner may overlap the first release liner near a center of the adhesive.

In an embodiment, the first release liner may include the first grasping portion, a first mid-portion, and the first contact portion, wherein the first grasping portion folds onto the first mid-portion at a first crease and the first mid-portion folds onto the first contact portion at a second crease. The first release liner may be configured such that the first grasping portion extends beyond a periphery of the adhesive device when the first grasping portion is unfolded away from the first mid-portion. The first release liner may be configured to be removed from the adhesive by a pulling motion of a user, wherein the at least one finger hole is configured to receive user's finger to facilitate grasping of the first grasping portion and pulling away the first contact portion towards an outer periphery of the adhesive device.

In an embodiment, the second release liner may be configured as a mirror image of the first release liner and may include a second grasping portion, a second mid-portion, and a second contact portion. In such an embodiment, the second release liner may be configured such that the second grasping portion folds onto the second mid-portion at a third crease and the second mid-portion folds onto the second contact portion at a fourth crease. The second release liner may be configured such that the second grasping portion extends beyond a periphery of the adhesive device when the second grasping portion is unfolded away from the second mid-portion. The second grasping portion may include at least one finger hole configured to receive user's finger to facilitate grasping of the second grasping portion and pulling away the second contact portion towards an outer periphery of the adhesive device to remove the second release liner from the adhesive by a pulling motion of a user.

In an embodiment, the two-piece release liner system may include a strip of tape configured to hold the first and second grasping portions together.

In an embodiment, an adhesive facing surface of the first contact portion and an adhesive facing surface of the second contact portion may be coated with a release coating configured for releasable attachment of the first and second contact portions to the adhesive. In some embodiments, at least a portion of the first and second grasping portions and/or at least a portion of the first and second mid-portions may be configured as a writeable surface to allow a user to write on the writeable surface using a marker or a pen.

In an embodiment, the first and second release liners may include a reinforced material for preventing tearing during removal.

In an embodiment, the skin barrier may be a convex barrier or a concave skin barrier.

In an embodiment, the adhesive device may be a medical adhesive device for attaching a suction tube, a drain tube, a vertical tube, a feeding tube, a horizontal tube, or an oral endotracheal tube to a user.

In an embodiment, the adhesive device may include a clamping mechanism configured to facilitate grabbing and removing the first and second release liners.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a distal side view of an ostomy barrier appliance according to an embodiment.

FIG. 2 is a schematic cross-sectional view of the ostomy barrier appliance of FIG. 1 with a two-piece split release liner partially open.

FIG. 3 is a front side view of one of the two-piece release liners unfolded.

FIG. 4 is a body side view of the ostomy barrier appliance of FIG. 1.

FIG. 5 is a distal side view of the ostomy barrier appliance of FIG. 1 with the two-piece split release liner partially open.

FIG. 6 is a body side view of the ostomy barrier appliance of FIG. 1 with one of the two-piece split release liners partially open.

FIG. 7A is a distal side view of the ostomy barrier appliance of FIG. 1 applied to a stoma and with the two-piece split release liner partially open.

FIG. 7B is a distal side view of the ostomy barrier appliance of FIG. 1 applied to the stoma and with one of the two-piece split release liners being removed.

FIG. 7C is a distal side view of the ostomy barrier appliance of FIG. 1 applied to the stoma and with the second of the two-piece split release liners being removed.

FIG. 7D is a distal side view of the ostomy barrier appliance of FIG. 1 applied to the stoma and with the two-piece split release liners fully removed and the barrier positioned on the stoma and secured to a patient.

FIG. 8 is a body side view of an ostomy barrier appliance including a tape for holding two release liners together according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

The present disclosure provides a two-piece split release liner system. The release liners can be part of an ostomy barrier appliance. The release liners can be applied to an ostomy barrier of a one-piece pouch system or a faceplate for a two-piece pouch system. The release liners can be used to align the barrier around a stoma and prepare a peristomal skin before removing each of the release liners and securing the barrier to the skin of a user. The two-piece release liner system may also be used for concave skin barriers or other skin contact adhesive devices, where it is beneficial to place the adhesive device against user's skin and check the position and sizing of the device before exposing an adhesive to the user's skin. For example, the two-piece release liner system may be used to protect an adhesive surface of medical devices of various shapes, such as suction tube attachment devices, drain tube attachment devices, vertical tube attachment devices, feeding tube attachment devices, horizontal tube attachment devices, and oral endotracheal tube fasteners, which are available from Hollister Incorporated, the Applicant of the present disclosure.

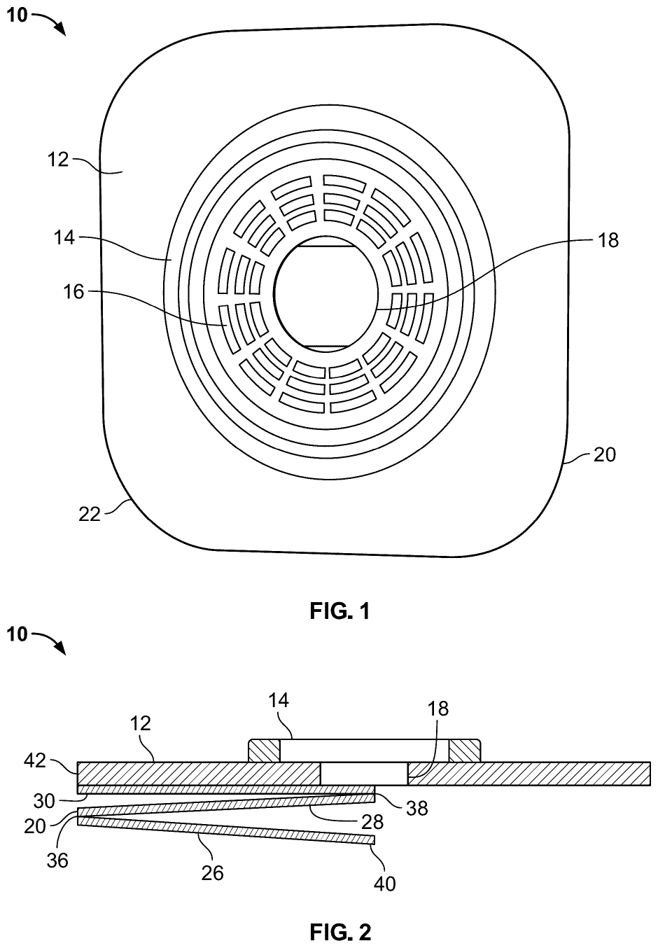

Referring now to the figures, FIGS. 1-7D show an ostomy barrier appliance 10 according to an embodiment. The illustrated appliance 10 is a two-piece appliance having a separate coupling member (not shown) that allows for attaching a pouch to the appliance 10.

FIG. 1 illustrates a distal view of an ostomy barrier appliance 10. The ostomy barrier appliance 10 can generally include a tape 12, a first portion 14 of a coupling for securing a pouch to a barrier, a skin barrier 16, an inlet opening 18 for receiving a stoma, a first release liner 20, and a second release liner 22.

The tape 12 may include an adhesive layer and a backing layer. The adhesive layer of the tape 12 can be formed from a suitable medical adhesive, such as an acrylic adhesive. The backing layer can be formed from a suitable material, such as a nonwoven material or a thin polymeric film. In another embodiment, the tape 12 can comprise a hydrocolloid adhesive and a film backing layer.

In some embodiments, the skin barrier 16 can include a backing layer laminated on the pouch-side surface of the skin barrier 16. The backing layer can be formed from a suitable heat sealable polymeric material, such that the backing layer can be heat sealed to the tape 12. The skin barrier 16 can be formed from a suitable skin-contact adhesive, such as hydrocolloid adhesives.

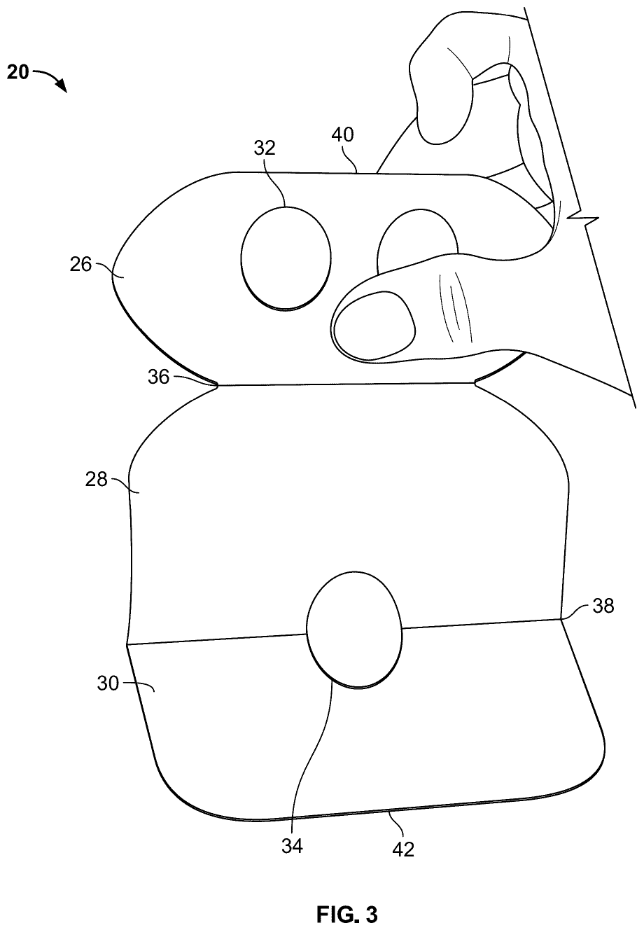

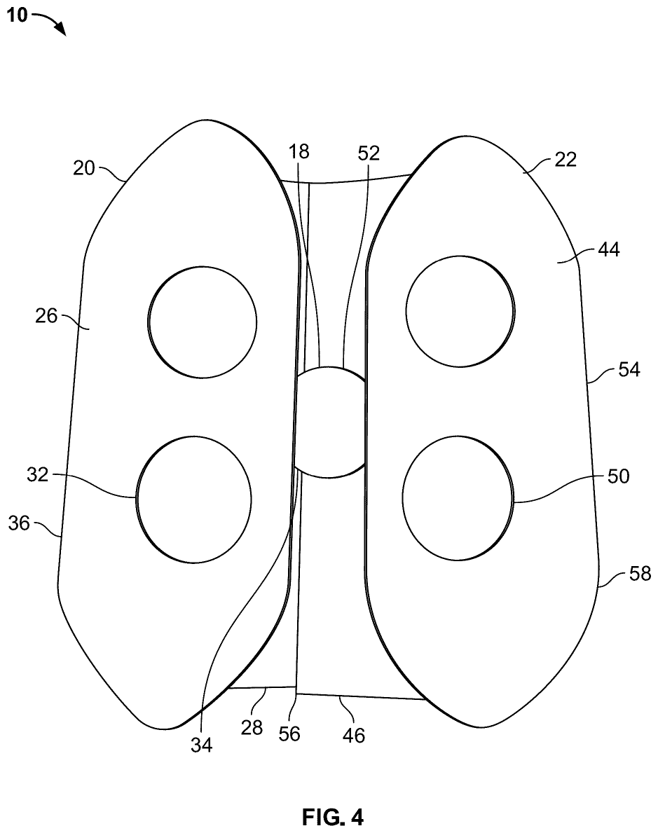

FIG. 2 illustrates a schematic cross-sectional view of the ostomy barrier appliance 10 with the first release liner 20 partially open and without the second release liner 22. FIG. 3 illustrates a front side view of the first release liner 20 unfolded. FIGS. 4 and 6 illustrate a body side view of the ostomy barrier appliance 10. The appliance 10 can include a two-piece split release liner comprising the first release liner 20 and the second release liner 22 attached to the body side of the tape 12 and skin barrier 16. The first release liner 20 can include a wing grasping portion 26, a mid-portion 28, a contact portion 30, a first crease 36, a second crease 38, a first end 40, and a second end 42 (FIG. 3). The first release liner 20 can further include one or more finger holes 32 and an opening 34 near the inlet opening 18. The first release liner 20 can be folded onto itself to form a z-like structure (FIG. 2). The wing grasping portion 26 can fold onto mid-portion 28 at the first crease 36 and the mid-portion 28 can fold onto the contact portion 30 at the second crease 38. The second end 42 can be located near an outer edge of tape 12, while the opening 34 can align with the inlet opening 18. The contact portion 30 may be configured to releaseably attach to the ostomy barrier appliance 10 and removed therefrom when pulled.

The second release liner 22 can be a mirror image of the first release liner 20 with a wing grasping portion 44, a mid-portion 46, a contact portion 48, a first crease 54, a second crease 56, a first end 58, and a second end 60 (FIGS. 4-6 and 7C). The second release liner 22 can also include one or more finger holes 50 and an opening 52 near inlet opening 18. The second release liner 22 can similarly be folded into a z-like structure as the first release liner 20.

The opening 34, 52 can be located between the mid-portion 28, 46 and the contact portion 30, 48 so that when the mid-portion 28, 46 and the contact portion 30, 48 are folded onto each other, the opening 34, 52 can align with the inlet opening 18. The release liners 20, 22 can be removed from the ostomy barrier appliance 10 by unfolding the wing grasping portion 26, 44 from the mid-portion 28, 46 and pulling on the wing grasping portion 26, 44 away from the ostomy barrier appliance 10. When the wing grasping portion 26, 44 is pulled, the contact portion 30, 48 may be pulled away from the ostomy barrier appliance 10 starting near or at the second crease 38, 56 towards the edge of the tape 12 thereby removing the release liners 20, 22 from the ostomy barrier appliance 10.

In an embodiment, the mid-portion 46 can overlap the mid-portion 28 (FIG. 4). This can allow for the adhesive to be fully covered by the release liners 20, 22 without a gap between them.

In an embodiment, the first and second release liners 20, 22 may be configured such that only adhesive facing surfaces of the contact portions 30, 48 are provided with a release coating. For example, the first and second release liners 20, 22 may be zone coated to apply a release coating, such as silicone release coating, only on the surfaces of the contact portions 30, 48 that are in contact with an adhesive. In such an embodiment, instructions and/or information related to the ostomy barrier appliance 10, for example, a cut-to-fit guide, may be printed on user facing surfaces of the first and second release liners 20, 22. The non-release coated surfaces of the first and second release liners 20, 22 may also be configured to allow a user to write with a marker or pen. In some embodiments, one or more surfaces of the first and second release liners 20, 22 may be coated with a writeable coating to provide writeable surfaces for users.

In an embodiment, a strip of tape 70 may be provided to hold the wing grasping portions 26, 44 together as shown in FIG. 8, so that the wing grasping portions 26, 44 may not be unfolded during storage or before application of the ostomy barrier appliance 10. The strip of tape 70 may be provided with an adhesive configured to releaseably attach to the wing grasping portions 26, 44 to hold them in place and easily removed by a user. The strip of tape 70 may have a user facing surface having a different color or a constrasting color to the release liners 20, 22 to facilitate removal of the strip of tape 70 by a user. In some embodiments, the user facing surface of the strip of tape 70 may include instructions and/or information printed thereon.

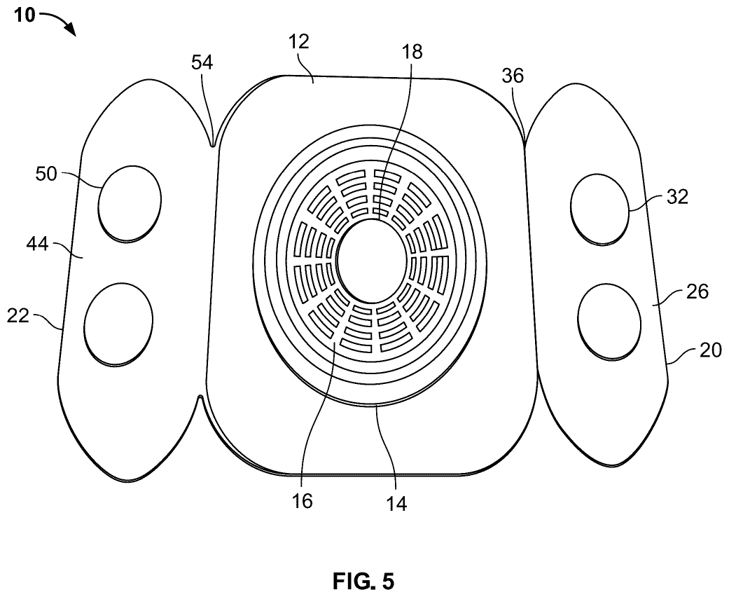

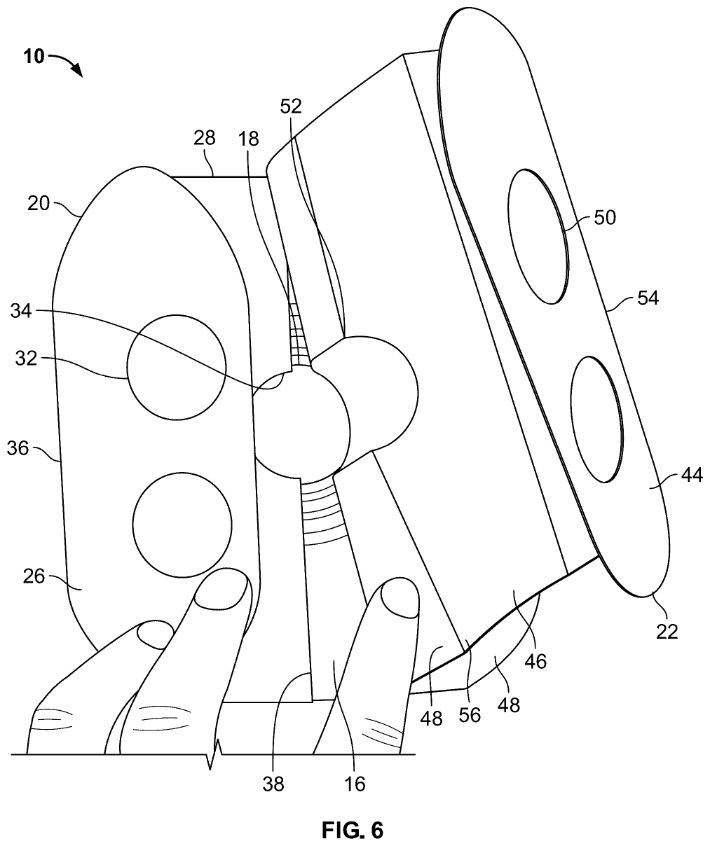

FIG. 5 illustrates a distal side view of the ostomy barrier appliance 10 with wing grasping portion 26, 44 unfolded. The release liners 20, 22 can be partially unfolded at the first crease 36, 54 to have the wing grasping portion 26, 44 extend past the edges of the tape 12.

FIG. 6 illustrates a body side view of the ostomy barrier appliance 10 with the second release liner 22 partially unfolded and pulled away proximate the second crease 56 to expose the skin barrier 16.

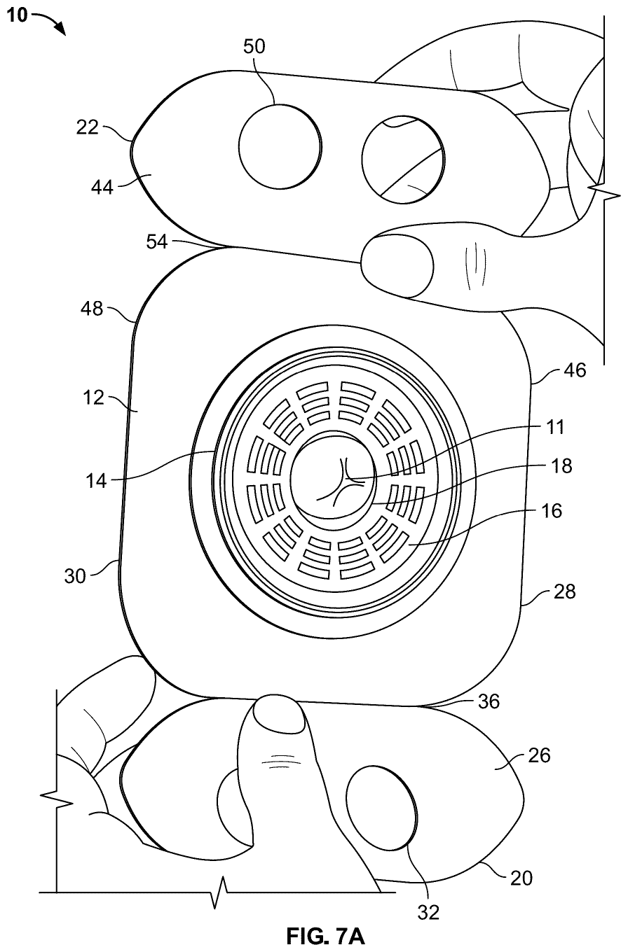

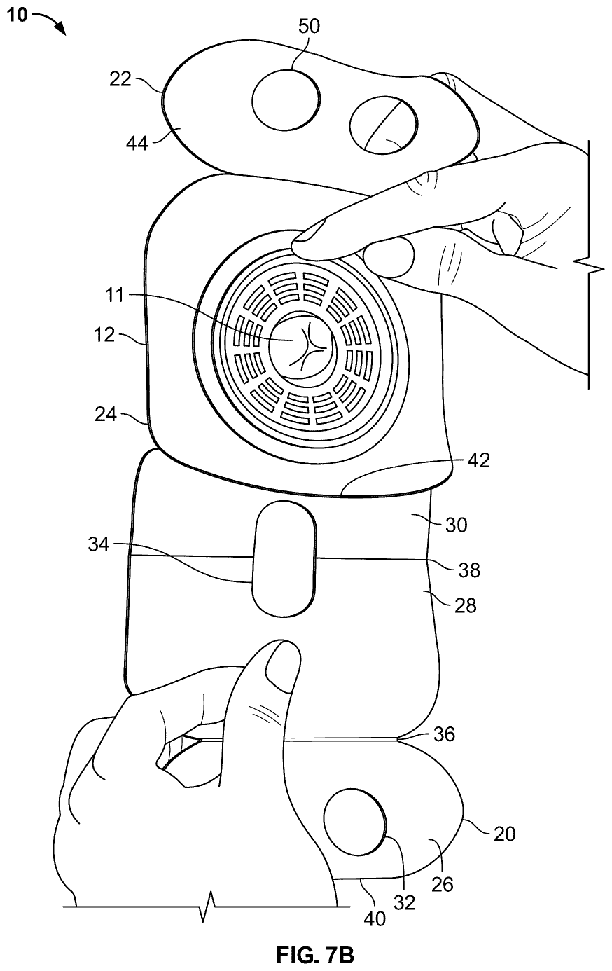

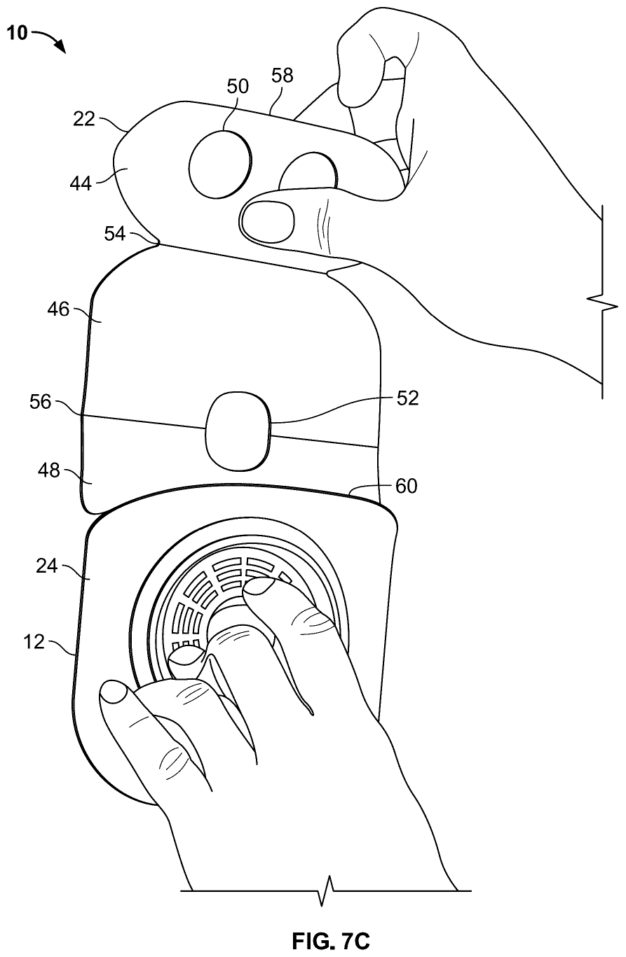

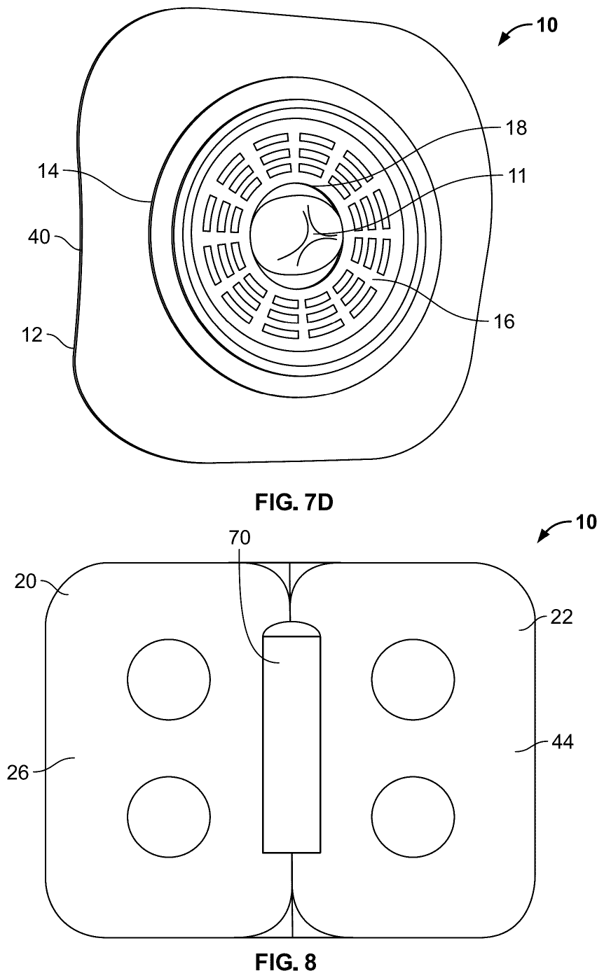

FIGS. 7A-7D illustrate a distal side view of the ostomy barrier appliance 10 as the release liners 20, 22 are being unfolded and removed from the ostomy barrier appliance 10 as it is applied around the stoma 11.

FIG. 7A shows the ostomy barrier appliance 10 with a skin barrier 16 surrounding a stoma 11 and the release liners 20, 22 being unfolded at first creases 36, 54 to expose the one or more finger holes 32, 50. The ostomy barrier appliance 10 can then be adjusted around the stoma 11 and smoothed out on the peristomal skin to facilitate attachment of the ostomy barrier appliance 10 to the user. The one or more finger holes 32, 50 can be used to grasp the wing grasping portion 26, 44 and pull on the release liners 20, 22 away from the inlet opening 18. Pulling on the release liners 20, 22 causes the second creases 38, 56 to be pulled away from the inlet opening 18 and expose the skin barrier 16 and the tape adhesive for attachment to the user's skin.

FIG. 7B shows the first release liner 20 being removed from the ostomy barrier 10 by pulling on the first release liner 20 away from the inlet opening 18 and towards the first end 40. The user, while pulling the first release liner 20, can adjust and/or smooth out the skin barrier 16 to facilitate attachment of the ostomy barrier appliance 10 to the peristomal skin. For example, the user may remove any air pockets or bumps in the skin barrier 10 while attaching the ostomy barrier appliance 10 to the peristomal skin.

FIG. 7C shows the first release liner 20 fully removed and the second release liner 22 being removed.

FIG. 7D shows the release liners 20, 22 fully removed and the ostomy barrier appliance 10 attached to a user's skin (not shown) and surrounding the stoma 11.

The split release liner system allows for sequential removal of two release liners after the ostomy barrier appliance 10 is placed against the peristomal skin. This provides a user an opportunity to correct any errors in placement and smooth out of the ostomy barrier appliance 10 to ensure substantially complete contact between the peristomal skin and the ostomy barrier appliance 10, thereby minimizing a risk of a premature leakage. The split release liner system may minimize air pockets and gaps and may provide improved adhesion between the ostomy barrier applaince 10 and the peristomal skin.

The release liner 20, 22 may be configured to extend sufficiently past the peripheral edges of the ostomy barrier appliacne 10 when unfolded and configured to allow a user to check for alignment with the stoma, press the ostomy barrier appliance 10 to user's skin, smooth out the peristomal skin to ensure contact with the ostomy barrier appliance 10, and then pull off the release liners 20, 22.

In an embodiment, the release liner 20, 22 can have a wing grasping portion 26, 44 that extends past the edges of the ostomy barrier 10 when unfolded. In an embodiment, the wing grasping portion 26, 44 may extend two inches past the edges of the ostomy barrier 10 when unfolded. In other embodiments, the wing grasping portion 26, 44 may extend based on the size of the ostomy barrier being used. The wing grasping portion 26, 44 can include finger holes 32, 50 to allow a user, with limited dexterity, to place one or two fingers through the release liner to pull the release liner off the barrier.

In an embodiment the release liner 20, 22 can have a reinforcing element to keep the release liner from tearing during removal and provide more control to the user.

In an embodiment, the ostomy barrier 10 and/or skin barrier 16 can be flat or convex.

In an embodiment, release liner 20, 22 can be coated so that its coefficient of friction to the peristomal skin is low. The low friction coefficient will aid in moving the ostomy barrier, with release liners still on, to the right position when it is pressed against the skin. Furthermore, the coating can also allow the user to write on the back of the release liner with a pen or marker.

In an embodiment, the ostomy barrier assembly may further include an accessory that may also be used to aid in removing the release liner. The accessory may have a clamping mechanism to grab the release liner and provide a more comfortable or easier grip solution. The accessory may also allow for shorter lengths of release liners than without the accessory. The accessory may have a method to fasten to the release liner and then be connected to a length of material before ending with a gripping mechanism.

In an embodiment, the second release liner 22, which extends over the first release liner 20, can be folded over the second release liner 20 to allow it to be more easily manufactured and take up less space when packed. The second release liner 22 can be folded back onto itself and be ready to use when needed.

In an embodiment, each of the first and second release liners 20, 22 may be configured to be the same size as the ostomy barrier 10. For example, for a 4 inch×4 inch ostomy barrier 10, each of the first and second release liner 20, 22 may be configured to have the substantially same size to the ostomy barrier 10, 4 inch×4 inch. In such an embodiment, each of the first and second release liner 20, 22 may be folded in half for attachment to the ostomy barrier 10, wherein wing grasping portions may be provided with zig-zag cuts and configured to expand beyond an outer periphery of the ostomy barrier 10 to function similar to the foregoing embodiments of the release liners 20, 22.

The release liner can additionally minimize the formation of air pockets and other discontinuities between the adhesive and the surface to which it is being applied. The user can not only ensure better alignment, but a more continuous application of the barrier to the stoma and surrounding tissue.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. An adhesive device for attachment to skin comprising:
an adhesive for attachment to skin; and
a two-piece release liner system for protecting the adhesive,
wherein the two-piece release liner system includes a first release liner and a second release liner, wherein the first release liner includes a first contact portion configured to be attached to the adhesive and a first grasping portion including at least one finger hole configured to facilitate removal of the first release liner by a user.

2. The adhesive device of claim 1, wherein the adhesive device is an ostomy barrier assembly for attaching an ostomy appliance to a peristomal skin surrounding a stoma comprising:
a skin barrier comprising the adhesive;
an inlet opening defined in the skin barrier for receiving the stoma; and
the two-piece release liner system attached to the skin barrier;
wherein the two-piece release liner system is configured to allow a user to expose the adhesive to the peristomal skin while the skin barrier is aligned with and surrounds the stoma.

3. The adhesive device of claim 1, wherein the two-piece release liner system is configured such that the first release liner covers about a half of the adhesive and the second release liner covers the other half of the adhesive, wherein a split between the first and second release liners is defined near a center of the adhesive.

4. The adhesive device of claim 2, wherein the two-piece release liner system includes a center opening aligned with the inlet opening.

5. The adhesive device of claim 1, wherein at least one of the first and second release liners is configured to extend past a periphery of the adhesive.

6. The adhesive device of claim 1, wherein the second release liner overlaps the first release liner near a center of the adhesive.

7. The adhesive device of claim 1, wherein the first release liner includes the first grasping portion, a first mid-portion, and the first contact portion, wherein the first grasping portion folds onto the first mid-portion at a first crease and the first mid-portion folds onto the first contact portion at a second crease, wherein the first release liner is configured such that the first grasping portion extends beyond a periphery of the adhesive device when the first grasping portion is unfolded away from the first mid-portion.

8. The adhesive device of claim 7, wherein the first release liner is configured to be removed from the adhesive by a pulling motion of a user, wherein the at least one finger hole is configured to receive user's finger to facilitate grasping of the first grasping portion and pulling away the first contact portion towards an outer periphery of the adhesive device.

9. The adhesive device of claim 7, wherein the second release liner is configured as a mirror image of the first release liner and includes a second grasping portion, a second mid-portion, and a second contact portion, wherein the second grasping portion folds onto the second mid-portion at a third crease and the second mid-portion folds onto the second contact portion at a fourth crease, wherein the second release liner is configured such that the second grasping portion extends beyond a periphery of the adhesive device when the second grasping portion is unfolded away from the second mid-portion.

10. The adhesive device of claim 9, wherein the second release liner is configured to be removed from the adhesive by a pulling motion of a user, wherein the second grasping portion includes at least one finger hole configured to receive user's finger to facilitate grasping of the second grasping portion and pulling away the second contact portion towards an outer periphery of the adhesive device.

11. The adhesive device of claim 9, wherein the two-piece release liner system includes a strip of tape configured to hold the first and second grasping portions together.

12. The adhesive device of claim 7, wherein an adhesive facing surface of the first contact portion and an adhesive facing surface of the second contact portion are coated with a release coating configured for releasable attachment of the first and second contact portions to the adhesive.

13. The adhesive device of claim 7, wherein at least a portion of the first and second grasping portions and/or at least a portion of the first and second mid-portions is configured as a writeable surface to allow a user to write on the writeable surface using a marker or a pen.

14. The adhesive device of claim 1, wherein the first and second release liners comprise a reinforced material for preventing tearing during removal.

15. The adhesive device of claim 2, wherein the skin barrier is a convex barrier or a concave skin barrier.

16. The adhesive device of claim 1, wherein the adhesive device is a medical adhesive device for attaching a suction tube, a drain tube, a vertical tube, a feeding tube, a horizontal tube, or an oral endotracheal tube to a user.

17. The adhesive device of claim 1, further comprising a clamping mechanism configured to facilitate grabbing and removing the first and second release liners.
